(19) United States
(12) Patent Application Publication
(10) Pub. No.: US 2009/0036925 A1
(43) Pub. Date: Feb. 5, 2009

(54) Device, Kit and Method For Intervertebral Stabilization

(75) Inventors: Giuseppe Sala, Desio (Milano) (IT); Paolo Guerra, Milano (IT); Piergiorgio Caserta, Milano (IT)

Correspondence Address: KILYK & BOWERSOX, P.L.L.C., 400 HOLIDAY COURT, SUITE 102, WARRENTON, VA, 20186, US

(73) Assignee: SINTEA BIOTECH S.P.A., Baranzate, MI (IT)

(21) Appl. No.: 12/067,511

(22) PCT Filed: Sep. 21, 2005
(86) PCT No.: PCT/IT2005/000540

§ 371 Date: Jul. 3, 2008

(51) Int. Cl. A61B 17/70; A61B 17/04

(52) U.S. Cl. 606/246; 606/264; 606/301; 606/305; 606/65

(57) ABSTRACT

An intervertebral stabilizing device adapted to stabilize two or more vertebral bodies one another. The device comprises two fastening elements adapted to be associated to two vertebral bodies being contiguous to each other and a junction element operatively connecting the two fastening elements. The fastening elements and the junction element gradually distribute the stresses both on the peduncles and the spinous bones of the adjacent vertebrae, so as to gradually and continuously stress the column length, thus avoiding sudden changes in load and stiffness in the borderline zones.

The present invention relates to a device for the intervertebral dynamic stabilization, adapted to correct the excessive mobility between two or more vertebrae while maintaining the normal gap between the latter. The present invention also relates to a medical kit for the intervertebral stabilization and an intervertebral stabilization method.

Different devices are known to dynamically stabilize two or more vertebrae between one another.

Some of these devices are of the ‘interspinous’ type, i.e. they comprise a pair of saddles, each to be ridingly associated to a spinous bone of two adjacent vertebral bodies; the saddles are associated to a same elastic body allowing relative motions between the vertebrae.

However, these devices considerably urge the spinous bones which are substantially cantilever stressed small-size beams. Therefore, there is the risk that the spinous bones may break.

On the other hand, other devices comprise peduncular beams or bars which are fastened to the peduncles of at least two adjacent vertebral bodies by means either of screws or bushes. These screws can be manufactured either as one piece or in several pieces, being elastically connected to one another. It has been seen that these devices have the fault in considerably stiffening the vertebral bodies being made integral to each other so as to cause a sudden stress discontinuity at the so-called ‘borderline zones’, i.e. the borderline areas on the unfastened vertebral bodies. Thereby, a fast degeneration of the sound vertebral bodies, being adjacent to the vertebral bodies fastened to each other by means of the same devices is caused.

The problem of the present invention is to provide a device for dynamic stabilization which solves the drawbacks mentioned with reference to the prior art.

These drawbacks and limitations are solved by a device for intervertebral dynamic stabilization in accordance with claim 1.

Other embodiments of the device according to the invention are described in the subsequent claims.

Further characteristics and the advantages of the present invention will be better understood from the description of preferred and non-limiting exemplary embodiments thereof as set for herein below, in which:

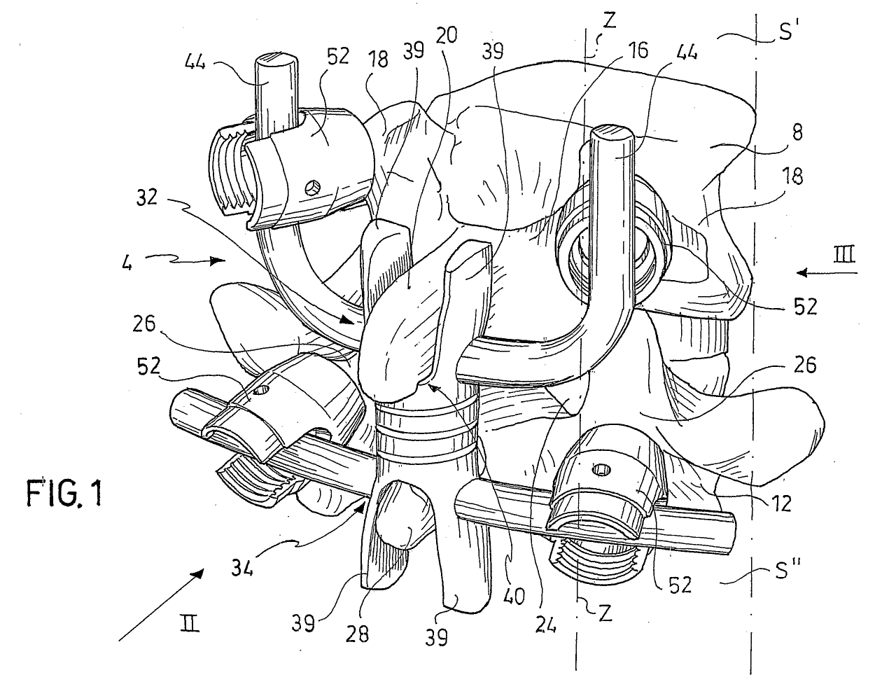

FIG. 1 shows a perspective view of a device for the intervertebral dynamic stabilization according to the present invention, in an assembly configuration between at least two vertebral bodies;

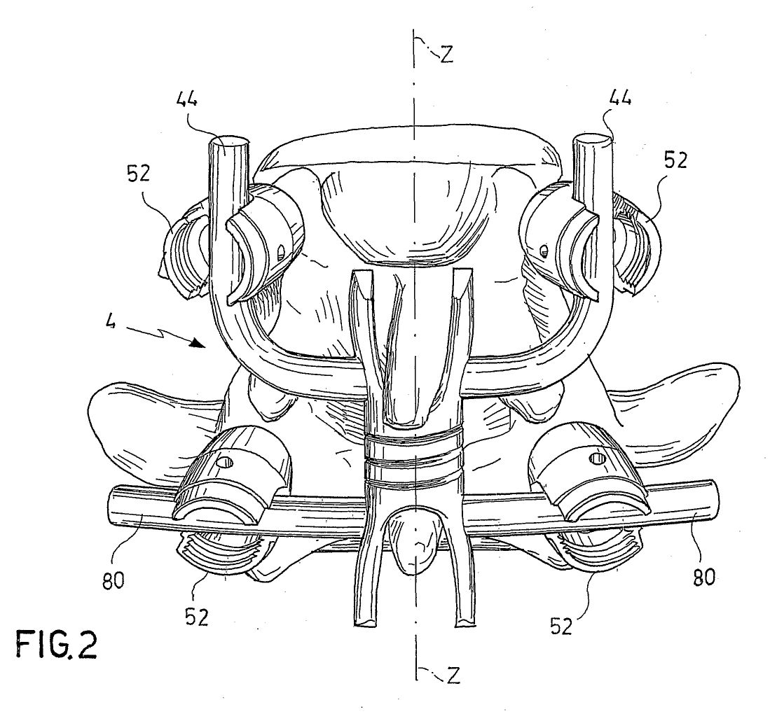

FIG. 2 shows a view of the device from FIG. 1, taken from the side of arrow II from FIG. 1;

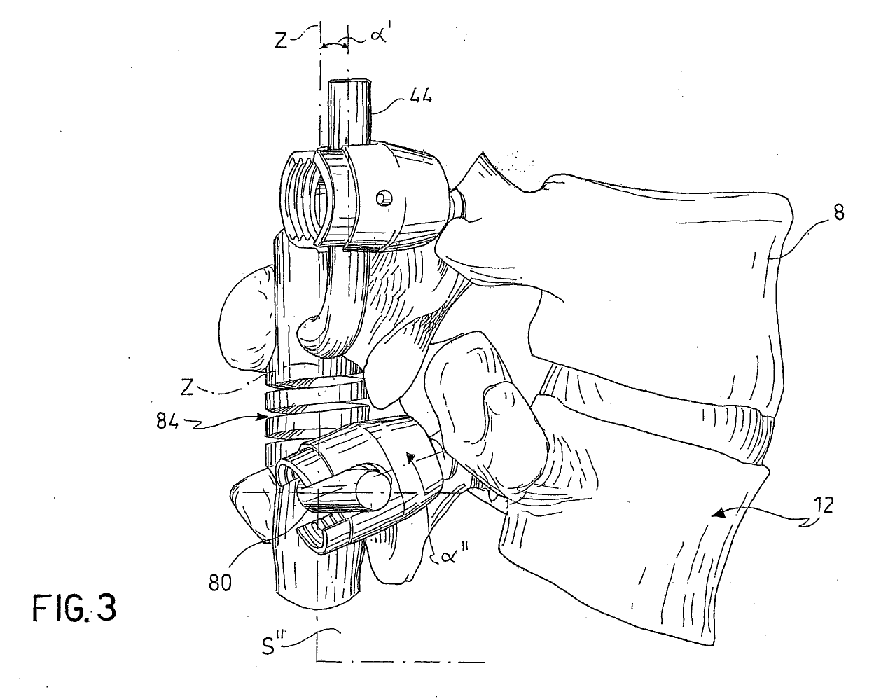

FIG. 3 shows a view of the device from FIG. 1, taken from the side of arrow III from FIG. 1;

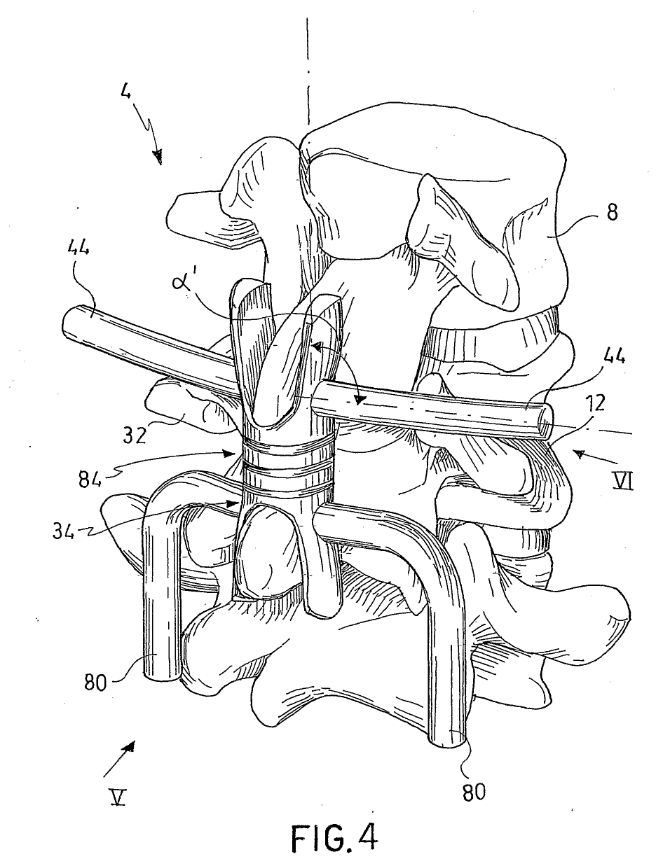

FIG. 4 shows a perspective view of a device for the intervertebral dynamic stabilization according to a further embodiment of the present invention, in an assembly configuration between at least two vertebral bodies;

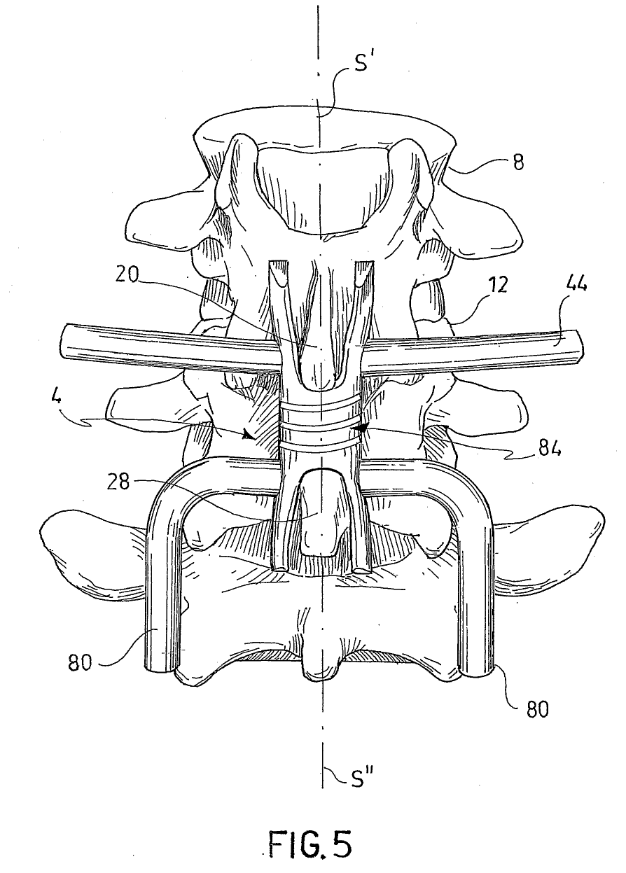

FIG. 5 shows a view of the device from FIG. 4, taken from the side of arrow V from FIG. 4;

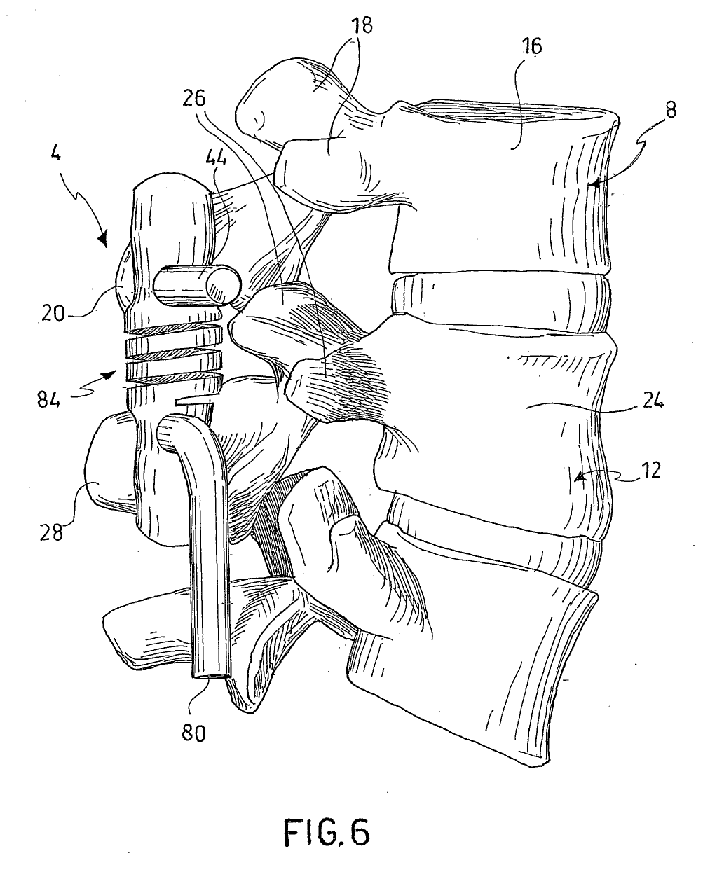

FIG. 6 shows a view of the device from FIG. 4, taken from the side of arrow VI from FIG. 4;

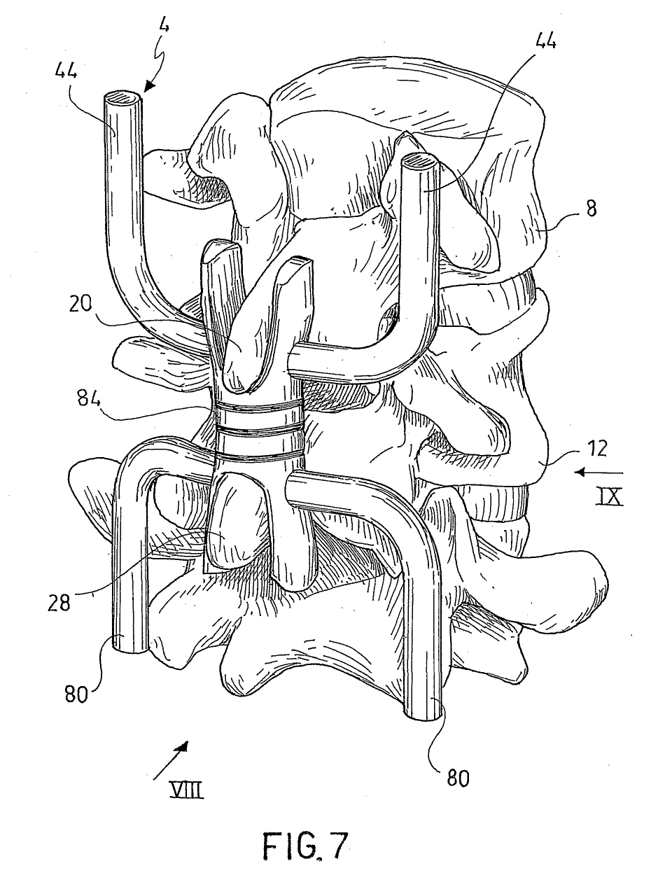

FIG. 7 shows a perspective view of a device for the intervertebral dynamic stabilization according to a further embodiment of the present invention, in an assembly configuration between at least two vertebral bodies;

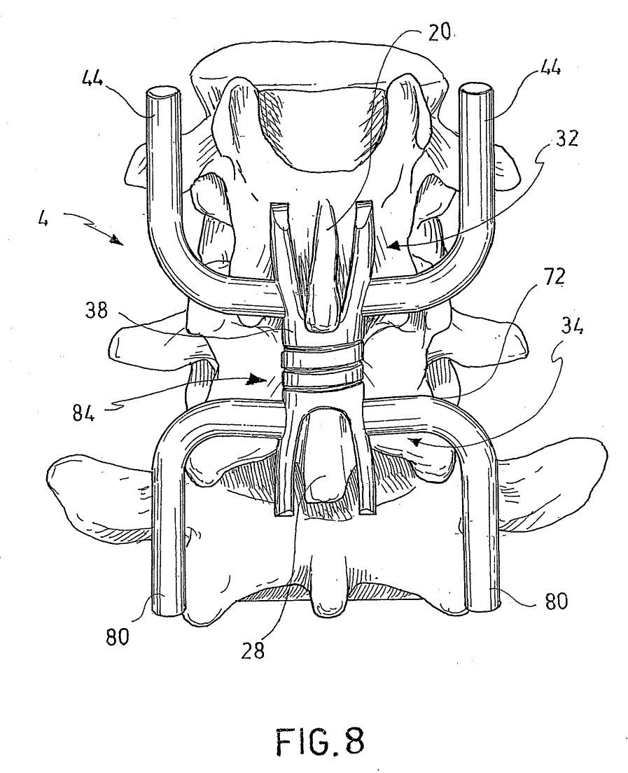

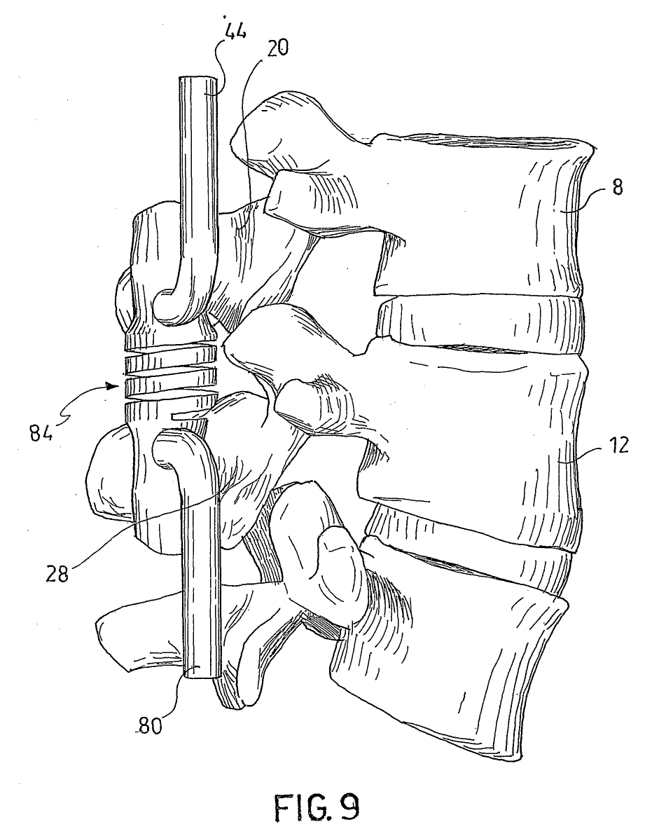

FIG. 8 shows a view of the device from FIG. 7, taken from the side of arrow VIII from FIG. 7;

FIG. 9 shows a view of the device from FIG. 7, taken from the side of arrow IX from FIG. 7;

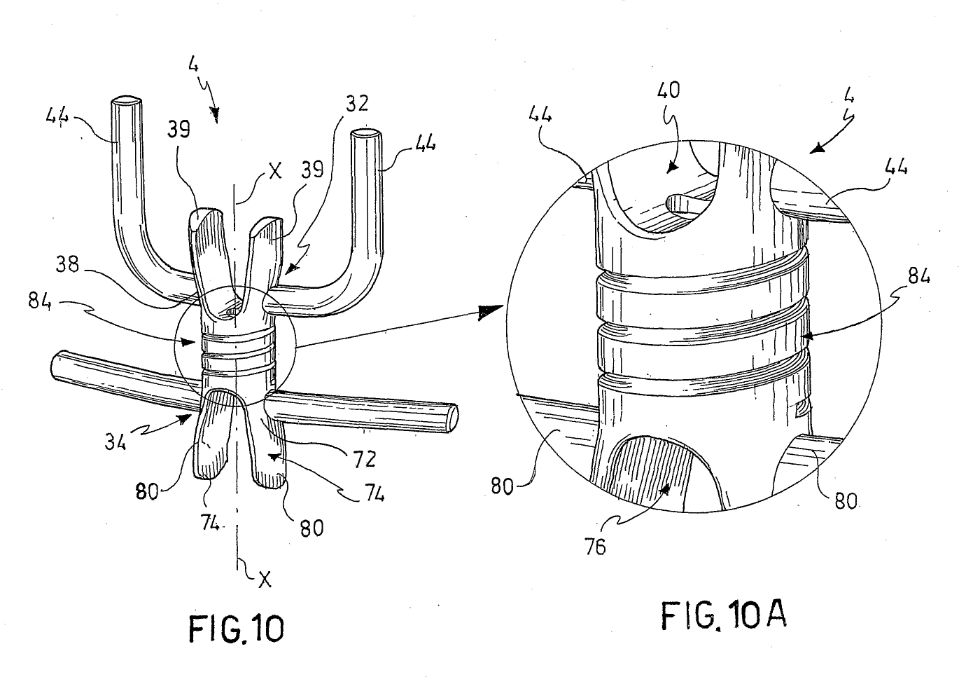

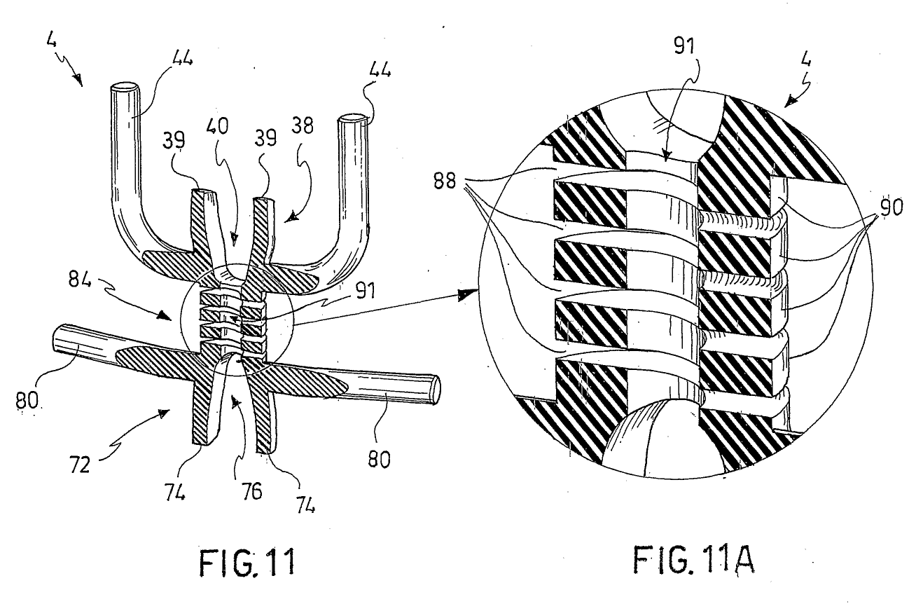

FIGS. 10 and 11 show a perspective view and a sectional view, respectively, of a device according to the present invention;

FIGS. 10A and 11A show enlarged details from FIGS. 10 and 11, respectively;

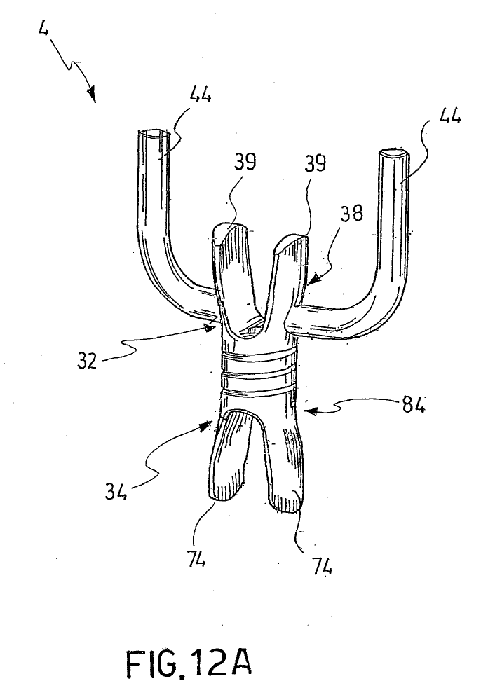

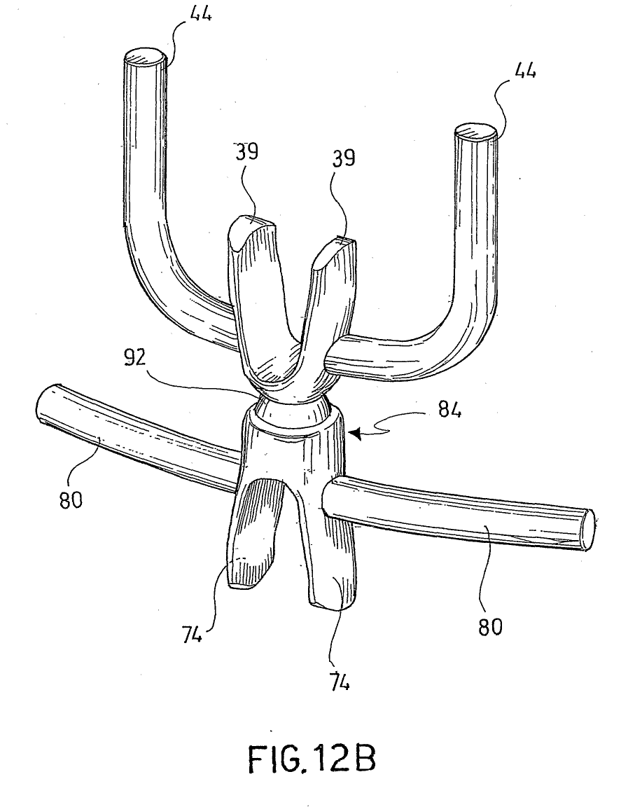

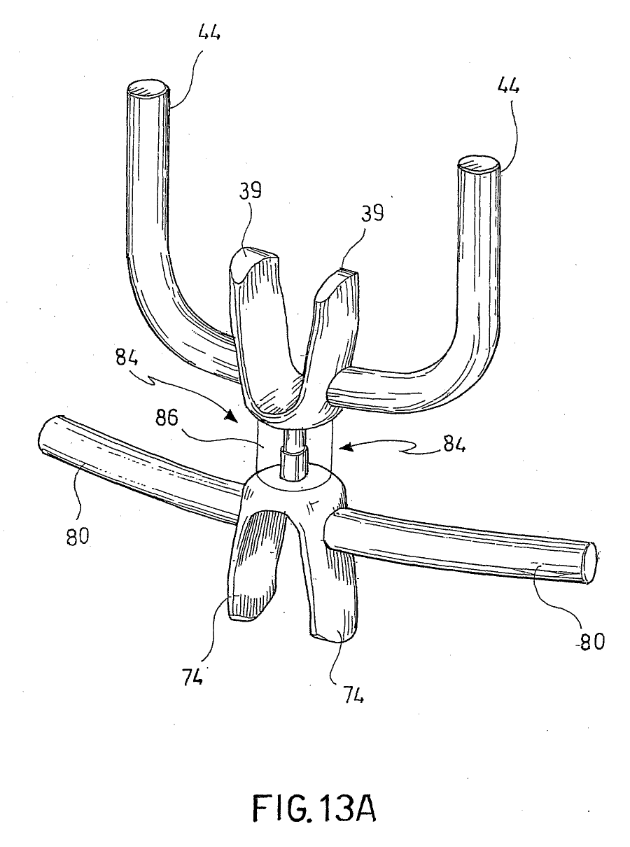

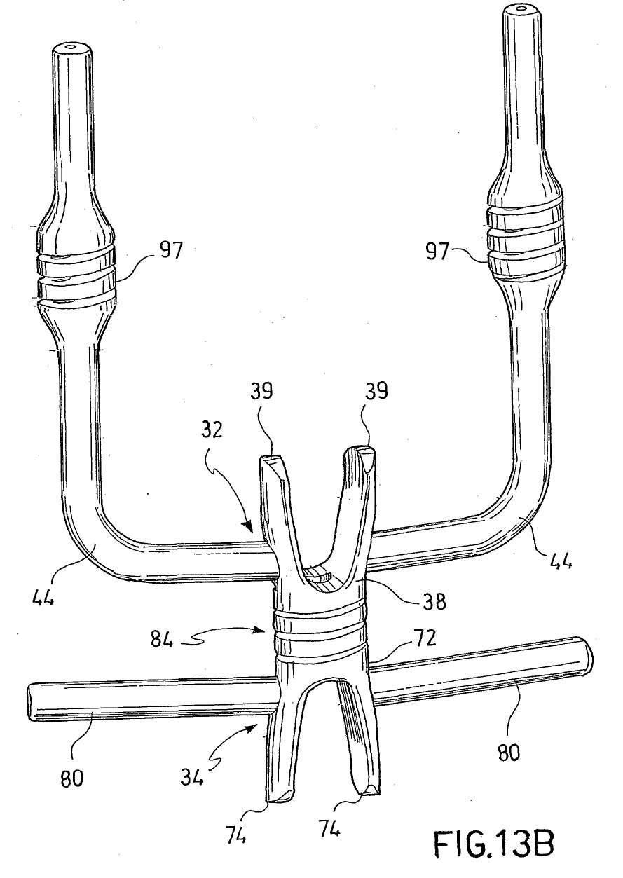

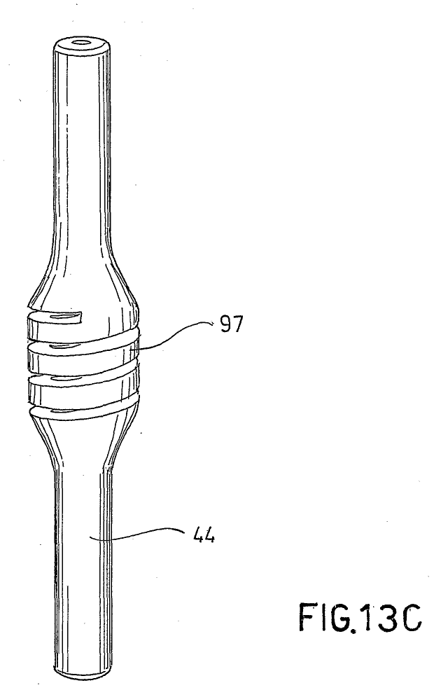

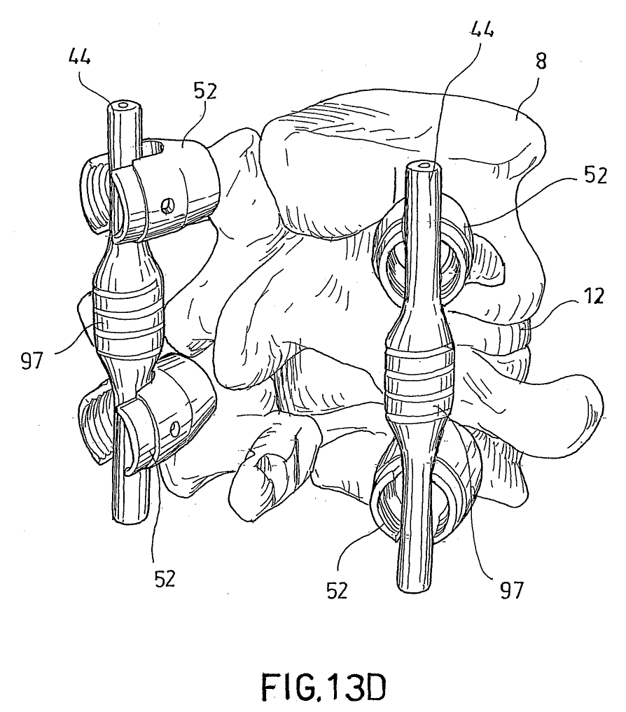

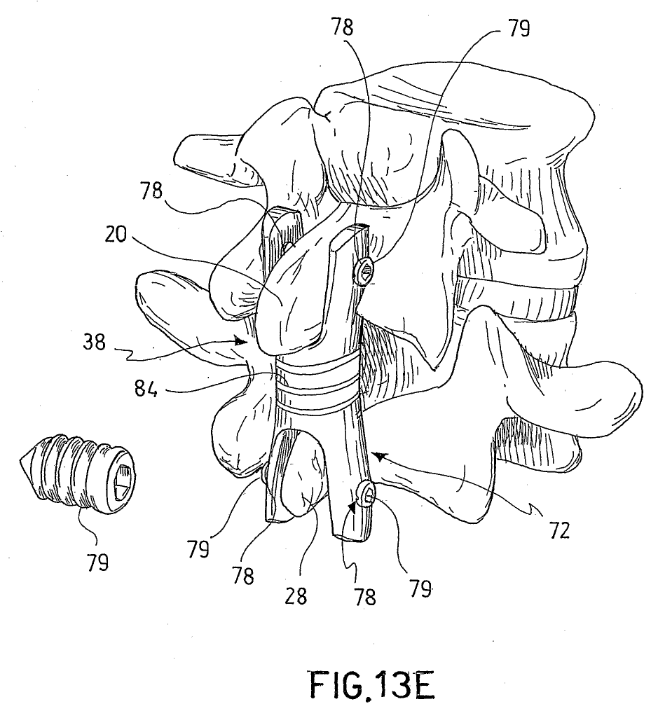

FIGS. 12A, 12B and 13A-13E show perspective views of further embodiments of devices for the intervertebral dynamic stabilization according to the present invention;

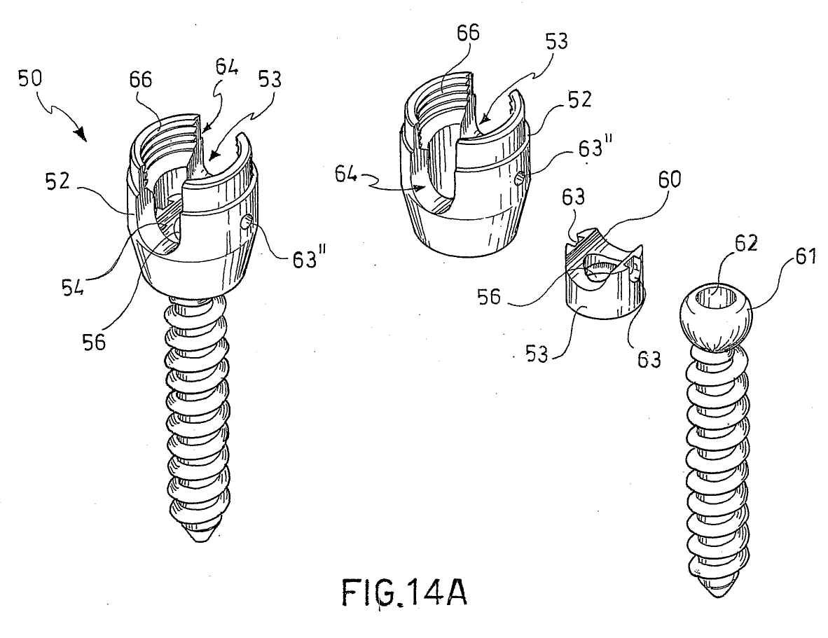

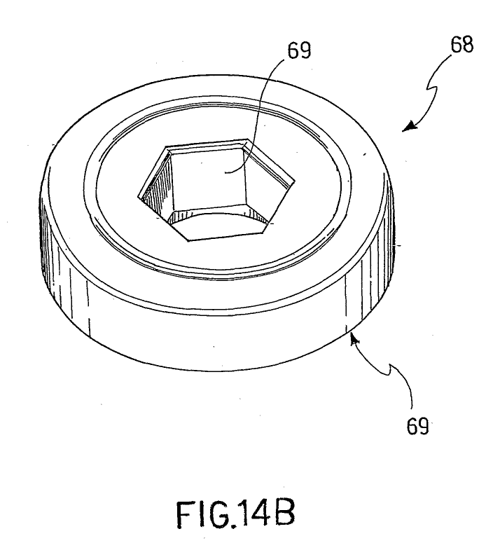

FIGS. 14A and 14B show perspective views of assembly elements of a device according to the present invention;

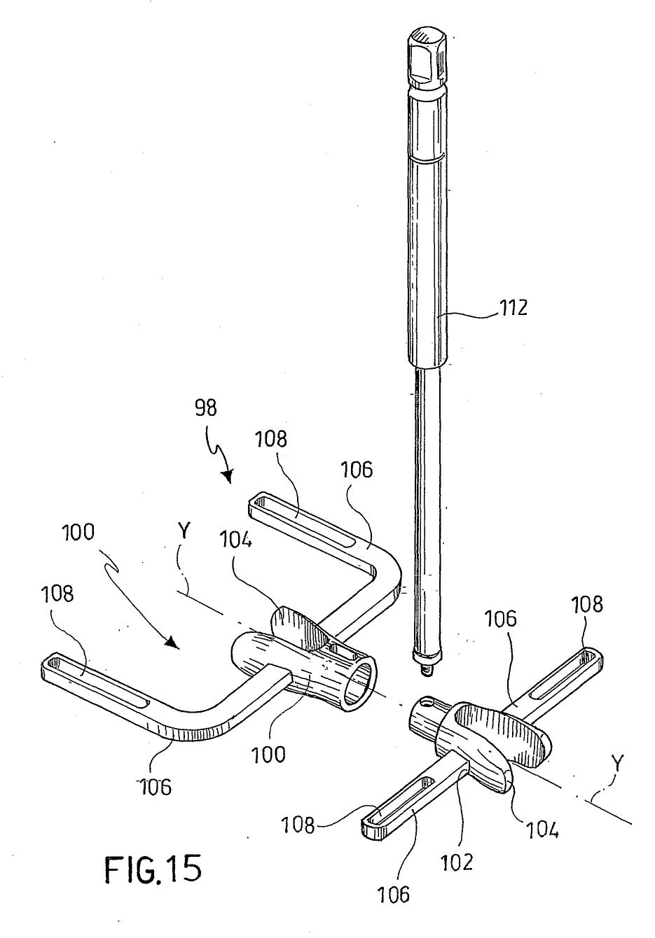

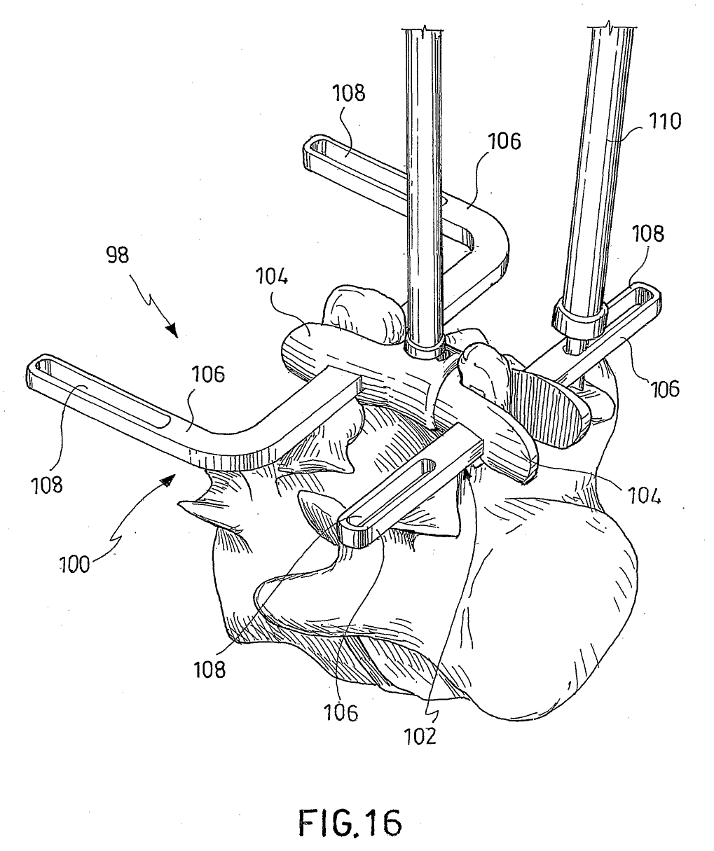

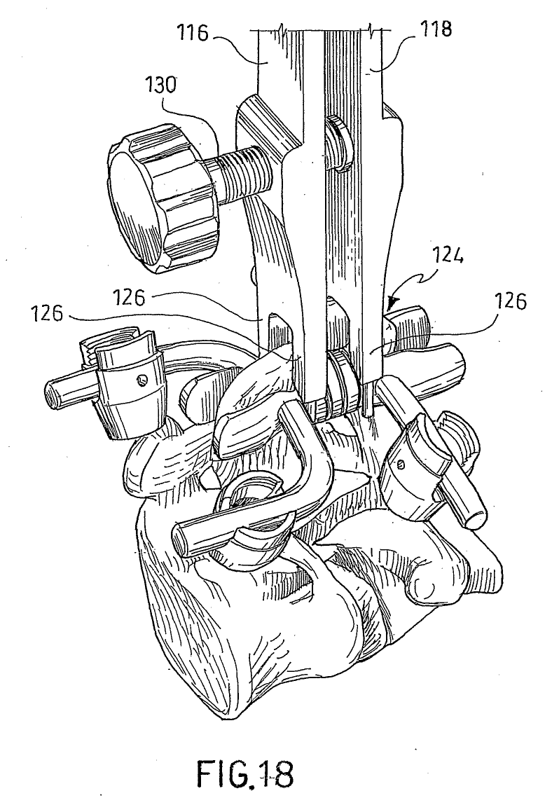

FIGS. 15, 16, 17A, 17B, 18 show inserting steps of a device for the intervertebral dynamic stabilization according to the present invention;

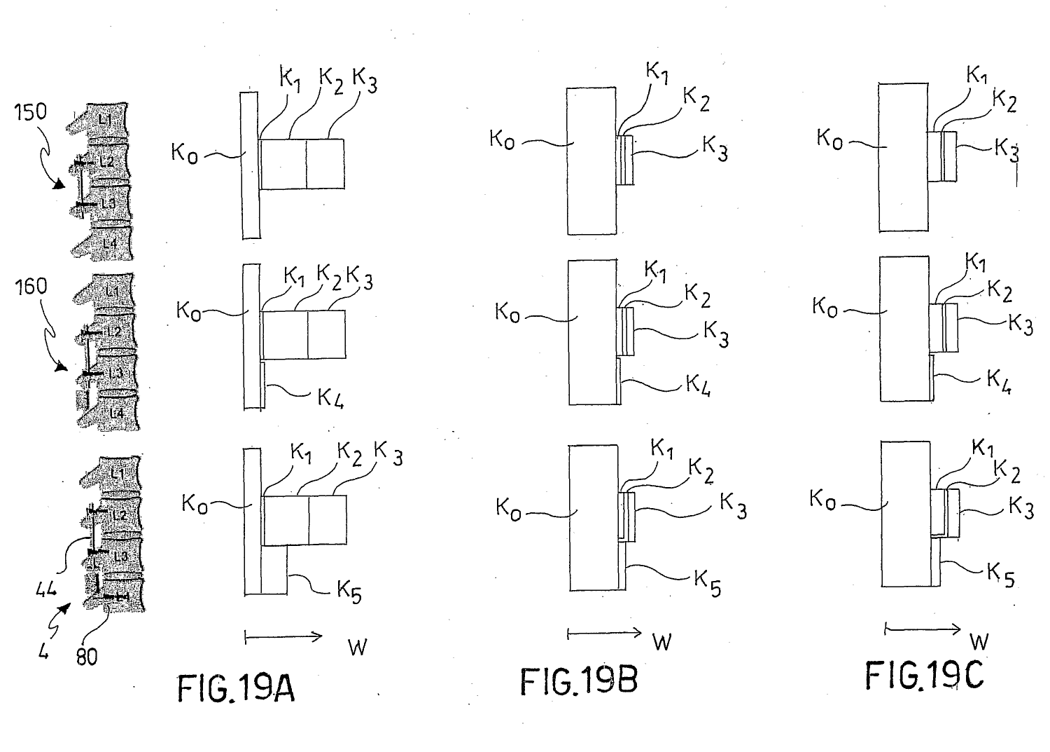

FIGS. 19A-19C show diagrams of compression, flexural and torsional stiffness, respectively, of a device according to the present invention compared to the prior art devices.

The elements or element parts in common among the embodiments described herein below will be indicated with the same reference numbers.

With reference to said figures, a device for intervertebral dynamic stabilization, adapted to be interposed between at least one first and one second vertebrae 8,12 being adjacent to each other, for example a cranial vertebra and a caudal vertebra, respectively, identifying an extending direction Z of the backbone length has been generically indicated with 4.

The first vertebra 8 comprises a first vertebral body 16 and a first pair of peduncles 18, being arranged on opposite sides relative to an anterior-posterior symmetry plane S′ of the same vertebra. The first vertebral body 16 comprises a first spinous bone 20, symmetrically arranged relative to said symmetry plane S′.

The second vertebra 12 comprises a second vertebral body 24 and a second pair of peduncles 26, being arranged on opposite sides relative to an anterior-posterior symmetry plane S″ of the same vertebra, preferably coincident with the symmetry plane S′ of the first vertebra 8.

The second vertebral body 24 comprises a second spinous bone 28, symmetrically arranged relative to said symmetry plane S″.

The device 4 comprises a first and a second fastening elements 32,34 adapted to be fastened to the upper and lower vertebrae 8,12, respectively.

According to an embodiment, the first fastening element 32 comprises a first coupling portion 38, adapted to be abutted on the first spinous bone 20. Preferably, said first coupling portion 38 has either a saddle or a ‘U’ configuration, comprising two branches 39, having incident portions, at a first groove 40 of the ‘U’ shape, and portions which are parallel to one another, at the ‘U’ arms. Thereby, the first coupling portion 38 may be fitted on the first spinous bone 20, for example according to a shape coupling, thus bringing the first groove 40 in abutment against the first spinous bone 20. In an assembly configuration, the bottom of the first groove faces the second vertebra 12, i.e. the branches 39 converge on the second vertebra 12.

The first groove 40 of said support portion has advantageously a thickness nearly equal to the spinal bone thickness, the thicknesses being measured relative to a direction which is perpendicular to the symmetry plane, so as to provide a shape coupling between the first groove 40 and the first spinous bone 20.

A first pair of fixing bars 44, preferably symmetrically arranged relative to the first coupling portion 38, branches from the first coupling portion 38 of the first fastening element 32.

Said first fixing bars 44 have for example a circular section and are curved so as to take a direction which is substantially parallel to the symmetry plane S′ of the first vertebra 8 and so as to intercept at least the first pair of peduncles 18.

Advantageously, the fixing bars 44 and the first coupling portion 38 are fastened to each other so that the stresses transmitted to the first fastening element are distributed both on the first spinous bone and on the first pair of peduncles.

Advantageously, the cross extension of the first pair of fixing bars 44 is such to intercept the opposite peduncles of the same vertebra; with cross extension is meant either the arm distance or length relative to a direction which is parallel to the symmetry plane S′ of the first vertebra 8.

Said first fixing bars 44 have for example a circular section and are for example oriented so that, on a plane perpendicular to the symmetry plane S′, are either angled or incident, by a first incidence angle α′ relative to Z-axis of the column length identified by the vertebrae. In other words, the fixing bars 44 are oriented so as to intercept the first pair of peduncles 18, so as to adjust the device 4 to the column length anatomy. The first incidence angle α′ ranges between 0 and 20 degrees and is preferably equal to 12 degrees.

Advantageously, the first fastening element 32 comprises a plurality of fastening screws 50, adapted to firmly fasten the latter to the vertebral bodies and particularly to the peduncles 18 of the first vertebra 8 and/or the vertebrae being adjacent to the first vertebra 8 on the opposite side of the second vertebra 12.

The fastening screws 50 comprise a bush 52, for example of a cylindrical shape and provided with a cavity 53. The cavity 53 is defined by an abutment 54 provided with a through hole 56, so as to have a circular ring shape as a whole. The abutment 54 is adapted to provide an end-of-stroke to the screwing of the screw into the bone.

Preferably, a retainer 58 provided with a circular milling 60 adapted to be abutted by a portion of said fixing bars 44 is housed within the cavity 53. The head 61 is also provided with a housing 62, for example of a prismatic hexagonal shape, in order to allow the screw to be screwed by means for example of an Allen wrench. The retainer 58 is advantageously provided with a pair of pockets 63, being diametrically opposite to each other. In an assembly configuration, the screw is inserted into the cavity 53 of the bush 52, thus bringing the head 61 in contact with the abutment 54. The retainer 58 is then inserted into the cavity 53, in contact with the head 61. Preferably, the bush 52 is provided with holes 63′ being diametrically opposite to one another along the side surface of the bush. Preferably, said holes 63′ are caulked so that this caulking also partially occupies the pockets 63. The caulking penetration into the pockets 63 ensures the axial locking of the head 61 between the abutment 54 and the retainer 58. The retainer 58 is provided with the through hole 56 in order to allow the screw head to have access to the housing 62 from the outside, i.e. from the side of the cavity 53 of the bush 52.

The fastening screw 50 is preferably of the self-tapping type so as to be capable of being directly screwed into the bone.

The bush 52 has a notch 64 extending throughout a diameter of the bush 52 and has a thickness which is not lower than the thickness of the bars, so as to allow the bars to be inserted thereinto.

The bush has an inner threading 66, being at least partially interrupted by the notch 64, on the opposite side of the through hole 56 of the retainer 58.

The screws 50 comprise a cap 68 of a cylindrical shape and provided with a threading 69 on the side surface thereof so as to be capable of being screwed onto the bush 52 after the fixing bars have been coupled therewith. Thereby, the cavities 53 of the bushes 52 are closed. The cap comprises for example a clamping hole 69 for inserting a wrench thereinto, for example either of the hexagonal type or the ‘torx’ type, in order to allow the same to be screwed.

According to an embodiment, the second fastening element 34 comprises a second coupling portion 72, adapted to be abutted against the second spinous bone 28. Preferably, said second coupling portion 72 has either a saddle or a ‘U’ configuration, comprising two branches 74, having incident portions, at a second groove 76 of the ‘U’ shape, and portions which are parallel to one another, at the ‘U’ arms. Thereby, the second coupling portion 72 may be fitted on the second spinous bone 28, for example according to a shape coupling, thus bringing the second groove 76 in abutment against the second spinous bone 28.

In an assembly configuration, the bottom of the second groove faces the first vertebra 8, i.e. the branches 74 converge on the first vertebra 8.

The second groove 76 of said support portion has advantageously a thickness nearly equal to the thickness of the second spinous bone, the thickness being measured relative to a direction which is perpendicular to the symmetry plane, so as to provide a shape coupling between the second groove 76 and the second spinous bone 28.

According to a possible embodiment, such as shown for example in FIG. 12A, at least one of said fastening elements, for example the second fastening element 34, is free of fixing bars, and the positioning and anchoring of the same to its respective vertebral body is ensured by the shape coupling between the second groove 76 and its respective spinous bone 28.

According to possible further embodiments, the anchoring between the spinous bones 20,28 and coupling portions 38,72 can be also ensured by means of small strings, passing around the spinous bones 20,28 and through suitable coupling holes provided on the branches 39,74. According to a further variant, the coupling portions can be fastened to the spinous bones by means of dowels 79 passing through the branches 39,74 and the spinous bones 20,28.

According to an advantageous embodiment, a second pair of fixing bars 80, preferably symmetrically arranged relative to the symmetry plane S″ of the second coupling element branches from the coupling portion of the second coupling element.

Said fixing bars 80 have for example a circular section and are for example oriented so that, on a plane which is perpendicular to the symmetry plane S″, are either angled or incident, of a second incidence angle α″ relative to the symmetry plane S″. In other words, the fixing bars 80 are oriented so as to intercept the second pair of peduncles 26 which are in a backer position relative to the spinous bone, so as to adjust the device 4 to the column length anatomy. This incidence angle α″ ranges between 0 and 20 degrees and is preferably equal to 12 degrees.

Advantageously, the fixing bars 80 and the second coupling portion 72 are fastened to each other so that the stresses transmitted to the second fastening element 34 are distributed both on the second spinous bone 28 and the second pair of peduncles 26.

Advantageously, the second coupling element also comprises a plurality of fastening screws 50, adapted to firmly fasten the latter to the vertebral bodies and particularly to the second vertebra 12.

Advantageously, the arm cross extension is such to intercept the opposite peduncles of the same vertebra; with cross extension is meant either the arm distance or length relative to a direction which is perpendicular to the symmetry plane.

The first and second fastening elements 32,34 are operatively connected to each other by means of a junction element 84, adapted to allow a relative rotary, translatory and flexural motion between the latter.

The junction element 84, according to an embodiment, as illustrated for example in FIGS. 10-12A, comprises a cylindrical body 86 provided with a plurality of notches 88, being arranged along the circumference of the latter. Said notches 88 have a radial depth which is lower than the radius of the cylindrical body 86, so as to provide a type of helical spring, provided with turns 90 for example of a helical shape.

Preferably, the cylindrical body is provided with a single notch helically arranged so as to provide a single continuous spiral.

The cylindrical body 86 is capable of allowing flexural motions between the first and second fastening elements 32,34 and hence between the vertebral bodies to which said fastening elements are associated, due either to the notches 88, or preferably to the single continuous notch. The stop for said relative motions is provided either by the abutment or pack closing condition of the element turns. Moreover, due to the notches, the element allows torsional motions relative to a rotation axis which is also a X-symmetry axis of the cylindrical body 86.

In order to allow a better flexibility of the junction element, the cylindrical body is provided with a central cylindrical hole 91. According to an embodiment, the central cylindrical hole 91 has a diameter which is equal to about ⅓ of the outer diameter of the cylindrical body.

The junction element 84 is preferably made as one piece, starting from a metallic cylindrical body being initially solid and then subjected to a processing step for example by means of electron discharge machining. According to further embodiments, the junction element may be obtained either by casting or by a machine-tool processing, preferably of the CNC-type.

The intervertebral stabilizing device 4 can be either totally or partially manufactured in titanium alloy, other types of metals or polymeric materials. For example, the junction element 84 can be manufactured in a different material compared to the connecting bars 44,80.

The ends of said element are advantageously integral to their respective fastening elements 32,34.

According to further embodiments of the present invention, such as shown for example in FIGS. 12B and 13, the junction element 84 can be provided by means of a joint of the ball type 92, adapted to allow relative rotary motions between the same elements. The joint of the ball type may also comprise a spring elastic element therein, so as to dampen the relative motion between the fastening elements 32,34 and also allow translatory axial motions along the extending direction of the vertebral column length involved.

According to a further embodiment, as illustrated for example in FIG. 13A, the junction element 84 may comprise a cylindrical body 86 containing a damper 96 therein, for example in polymeric material, optionally filled with saline therein.

The device according to the present invention may comprise various variant embodiments. For example, only one of the fastening elements can be provided with fixing bars, such as illustrated for example in FIG. 12A. The fixing bars can be both transversally and axially oriented, both at the first 32 and second 34 fastening elements. Furthermore, as illustrated in FIGS. 7 and 8, both the fastening elements may comprise bars which are longitudinally arranged on the side of their respective branches 39.

Moreover, the connecting bars can be extended so as to involve a plurality of vertebral bodies; for example, the bars of the first vertebra may intercept two or more vertebrae being adjacent to the first vertebra on the opposite side of the second vertebra, so as to stabilize a column length comprising three or more vertebrae being consecutive to one another.

The connecting bars, such as shown for example in FIG. 13B, may comprise flexible elements 97 similar to the junction element 84, preferably comprising a cylindrical body provided with a continuous helical groove and a central hole so as to ensure further flexibility to the connecting bars, as well as a better adjustment of the connecting bars to the column length morphology.

Advantageously, the device according to the present invention is implanted by using some tools.

As shown for example in FIGS. 15-18, there is provided a template tool 98 comprising a first and second measuring elements 100,102 each adapted to be interfaced with the spinous bones 20,28 of the two adjacent vertebral bodies 8,12.

In fact, each of said measuring elements 100,102 comprises a saddle portion 104, adapted to be fitted on its respective spinous bone, and a pair of slotted links or arms 106, being symmetrically arranged relative to said saddle portion 104, so as to intercept the peduncles of their respective vertebral bodies.

Each slotted link 106 is provided with a groove 108, adapted to allow either a point of a punch 110 or of a marking tool to pass through. The punch can be provided with a point adapted to scratch the cortex of the vertebral body at the peduncles. The marking tool can be provided for example with a point adapted to mark with a dot, being for example coloured, the cortex of the vertebral body at the peduncles.

The two measuring elements 100,102 can be axially adjusted to each other, along a mutual extension axis by means of a screwdriver 112. Preferably, a graduated scale is inserted at the connecting portion between the two measuring elements 100,102, in order to provide a direct measuring of the size of the device that best suits the column length.

A further tool for inserting the device is represented by a gripper 114 having a pair of grip means 116,118. The grip means 116,118 are slidingly associated to each other at a first end 120 by the interposition of a pivot 122, whereas, at a second end 124 opposite to said first end 120, each is provided with a pair of tines 126 adapted to grasp the device at the junction element 84.

The gripper 114 is provided with a gauged screw 130 adapted to adjust the axial distance between the two grip elements 116,118 which can relatively slide on each other along the pivot 122 in common, at a central portion 128, included between said ends 120,124.

The gripper 114 allows the device 4 to be grasped from the side of the junction element by locking the tines 126 between the junction element and the fixing bars 44,80.

The technique for inserting the device according to the invention will be now described herein below.

Particularly, the device 4 is preferably pre-assembled, i.e. the fastening elements 32,34 and the junction element are already pre-assembled to each other so as to form a single device 4.

The pre-assembly can be preferred both in the embodiment with a spring junction element 84, and in the embodiments with a joint either of the ball or damping type.

Advantageously, before proceeding with the device insertion, the template tool 98 is used by approaching this tool to the column length to be stabilized.

The template tool 98 is firstly fitted on the spinous bones of the vertebrae to be stabilized, thus bringing the saddle portions 104 in contact with the spinous bones; then, the axial position between the two measuring elements 100,102 is adjusted.

The marking of the vertebra peduncles on which the device 4 will have to be subsequently fastened is then carried out. The marking can be carried out with the aid either of a drift 110 or a marker, by inserting the latter through the grooves 108 provided on the slotted links 106.

The template tool 98 which can thus provide the indication of the size of the device to be implanted is then removed. With dimension or size is meant the distance between the two grooves 40 and 76 in a resting configuration of the junction element 84. Thereby, it is possible to select the device 4 with the size which is more easily adapted to the morphology of the column length to be stabilized.

The screw positioning on the peduncles and the screw screwing onto the peduncles are carried out at the markings so as to lock the bushes in position. After the screws have been screwed, the bushes may advantageously rotate relative to the peduncles, so as to orientate the notches 64 of the bushes parallel to the final arrangement which the connecting bars of the fastening devices will have to take; for example, longitudinally in the case of the first locking element and transversally in the case of the second locking element.

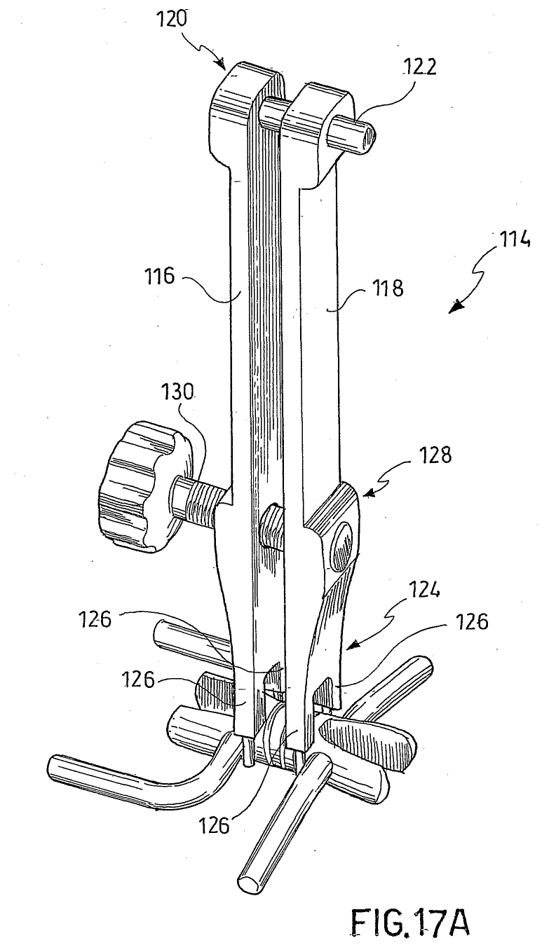

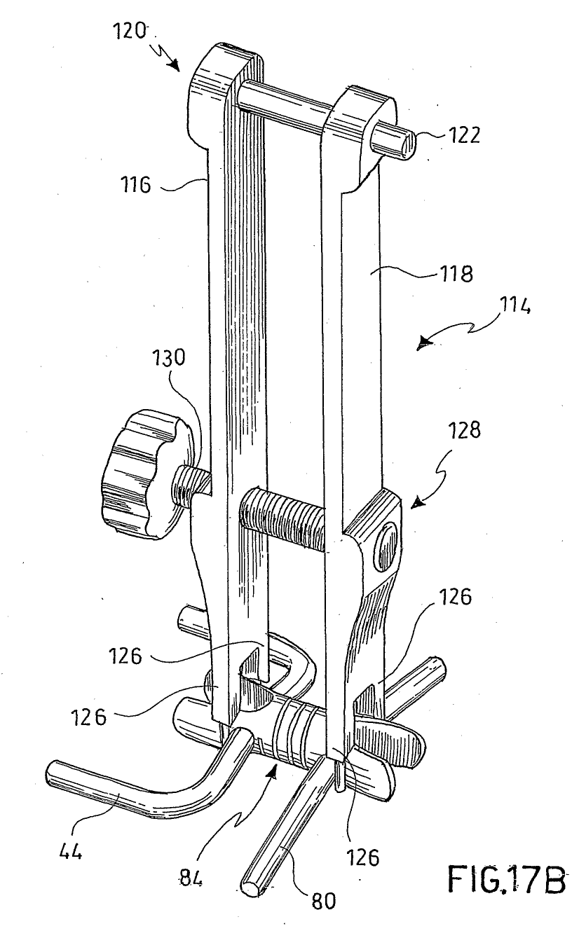

Therefore, with the aid of the gripper 114, the device 4 is grasped and positioned near the vertebrae to be connected, by inserting the bars of the first and second fastening elements 32,34 into their respective notches of the bushes already fastened to the peduncles. Particularly, the connecting bars are placed in abutment on the millings 60 of the retainers 58. The device grasping with the gripper, as illustrated in FIG. 17A, may be performed by locking the tines 126 between the coupling portions 38,72 and their respective fixing bars 44,80, so as to allow the junction element to be compressed in order to position the latter among the spinous bones. According to a further embodiment, as illustrated in FIG. 17B, the tines can be fitted on suitable housings or slots provided on the junction element in a length included between the fixing bars 44,80.

At the same time, the device saddles are inserted onto the corresponding spinous bones, thus bringing the groove bottom in abutment against the spinous bones. Particularly, with the removal of the gripper 114, the junction element is axially preloaded, i.e. along Z-axis, in compression, so as to ensure the contact between the saddles and their respective spinous bones.

The bush notches advantageously allow to modify the relative position between the bars and the bushes, so as to be able to adapt the device to the specific physiology of the column length.

After the proper position of the fastening elements has been set, the final locking of the device bars is then carried out by inserting and screwing the caps 68 onto the bushes 52, by means of a suitable clamping wrench inserted into the hole 69. Following this clamping, the cap 68 thrusts the connecting bar against the retainer 58, in an approaching direction to the spinous bone. The head 61 is further rotatably locked by friction against the retainer 58.

The operation of the device according to the invention will be now described herein below.

After it has been fastened to at least two vertebral bodies being adjacent to each other, the device according to the invention allows relative motions between the vertebral bodies connected to each other. These motions are of the axial, flexural and torsional type and are ensured by the yielding of the junction element.

Particularly, the device allows the axial, flexural and torsional stiffness to be uniformly and gradually distributed along the column length involved, without sudden changes which could cause excessive stresses in the so-called borderline areas, as illustrated in FIGS. 19A-19C. Particularly, FIGS. 19A-19C show diagrams of compression, flexural and torsional stiffness, respectively, of a device 4 according to the present invention as compared with further possible variants of devices marked with references 150, 160. The possible device 150 only comprises stiff connecting bars being fastened to the peduncles, whereas the possible device 160 comprises stiff connecting bars among the peduncles which bars are side by side with elastic elements between the spinous bones, the elastic elements being mechanically unfastened by the connecting bars, i.e. there are no mechanical connections between the bars and the elastic elements, therefore the bars and the elastic elements are arranged in succession to each other.

The stiffness is represented in the form of histograms extending along a direction W in common. The value k0 represents the stiffness of the column length free of any stabilizing device.

In all the diagrams, the values k1,k2,k3 represent the stiffness contribution provided by peduncular bars manufactured in polymer, titanium and steel, respectively, in the sizes usually employed in the art. The values marked with k4 represent the contribution provided by the stabilizing device 160 comprising stiff connecting bars between the peduncles, side by side with elastic elements between the spinous bones, in which the elastic elements are mechanically unfastened by the connecting bars. The values marked with k5 represent the stiffness contribution provided by the stabilizing device according to the invention. It should be noted that the stabilizing device according to the invention, compared to the other devices, always ensures the less discontinuity among the stiffness of the column length and hence a gradual stress distribution both between the vertebrae directly connected to each other, and between the vertebrae adjacent to each other in the borderline zones.

The fastening elements directly connect the coupling portions to the fixing bars. Thereby, the stresses on the column length are uniformly and gradually distributed both on the spinous bones and the peduncles.

The compressed preload of the junction element, following the insertion of the same between the vertebral bodies, ensures a continuous contact between the fastening elements and the spinous bones. Thereby, the loads are always also distributed on the spinous bones, besides on the peduncles.

The stiffness of the column length involved with the device, as compared with the physiology of the sound column length, is also gradually and uniformly modified without sudden changes occurring both in the axial, flexural and torsional stiffness. By extending the fixing bars, the loads of the column length, as well as its respective stiffness, can be distributed on an increasing number of vertebrae, so as to respect the column length physiology as much as possible. Thereby, sudden changes in stiffness at the borderline zones or areas, i.e. the column zones adjacent to the device, do not occur.

As may be appreciated from what has been described, the described device allows one to overcome the drawbacks occurred in the prior art.

Particularly, the device allows a uniform load distribution between the peduncles and the spinous bones of the vertebral bodies.

Thereby, the borderline zones are loaded gradually, without the presence of sudden load changes.

In fact, each stiffness of the vertebral bodies gradually change from one another, without sudden discontinuities.

Furthermore, the spinous bones are suitably loaded, i.e. they are not overloaded because they are not intended to suffer all the stresses transmitted between two contiguous vertebrae; the risk of dangerous breaks of the latter is thus avoided. In other words, the load burdening the vertebral bodies is not totally discharged on the spinous bones, but it is suitably distributed between the spinous bones and the peduncles.

The loads burdening the spinal length related to the device according to the invention, are advantageously distributed both on the spinous bones and on the peduncles of the vertebral bodies; thereby, sudden, dangerous changes in the stress distribution on the vertebrae adjacent to said spinal length, the so-called borderline zones, do not occur.

The device may be also easily implanted on a spinal length thanks to the possibility of adjustment offered by the lock bushes. This adjustment is both axial, i.e. either a sliding or a relative translation between the connecting bars and the bush notches, and angular, i.e. an orientation of the bush notches.

The presence of the junction element provided with helical-course grooves ensures both a flexural and torsional proper stiffness so as to ensure but also support the normal flexural and torsional motions of the column.

The device according to the invention is capable of maintaining the kinematics of the column segment to which it is connected and at the same time it is capable of providing an elastic support and acting as a damper being interposed between the spinous bones of the vertebral bodies of the same segment.

The device ensures the main physiological functions of the intervertebral disks, such as the correct kinematics for example of the rachis and the ability of transferring the loads and dampening the dynamic stresses.

The joint allows movements and bending and also acts as a shock absorber.

The interaction between the peduncular bars, fastened to the peduncles of the vertebral bodies, and the joint associated to the spinous bones ensures a proper and gradual load distribution not only on the column length related to the device but also on the borderline zones, i.e. on the adjacent vertebrae.

With this interaction the spinous bones are not overloaded and at the same time a part of the loads is absorbed by the adjacent and thus sound vertebral bodies.

The device does not cause arthrodesis, thus always ensuring the correct kinematics between the vertebral bodies.

Those skilled in the art, aiming at satisfying contingent and specific needs, will be able to carry out several modifications and variants to the intervertebral devices described above, all of them being contemplated within the scope of the invention such as defined by the following claims.

1-34. (canceled)

35. An intervertebral stabilizing device, adapted to be interposed between at least a first and a second vertebrae being adjacent to each other,
comprising a first fastening element adapted to be associated to the first vertebra
and a second fastening element adapted to be associated to the second vertebra
the first and second fastening elements each comprising a coupling portion adapted to be coupled with a spinous bone of the first and second vertebrae respectively,
the first and second fastening elements being operatively connected to each other by means of a junction element adapted to allow relative motions between the fastening elements characterized in that
at least one of the fastening element comprises fixing bars adapted to be fastened to peduncles of at least one of the first and second vertebrae so as to discharge the forces exchanged between the first and second vertebrae both on the spinous bones of the vertebrae and the peduncles of at least one of the vertebrae

36. The intervertebral stabilizing device according to claim 35, wherein the first and second fastening elements comprise first and second fixing bars respectively, adapted to be fastened to their respective peduncles of the first and second vertebrae

37. The intervertebral stabilizing device according to claim 35, wherein the first coupling portion has either a saddle- or a ‘U’-shaped configuration, comprising two branches so as to be fitted on the first spinous bone according to a shape coupling.

38. The intervertebral stabilizing device according to claim 37, wherein to the first coupling portion is associated the first pair of fixing bars so that the stresses transmitted to the first fastening element are distributed both on the first spinous bone and the first pair of peduncles

39. The intervertebral stabilizing device according to claim 35, wherein the first fixing bars are oriented so that, on a plane perpendicular to a symmetry plane of the first vertebra they are angled according to a first incidence angle relative to a vertical axis of the first vertebra so as to intercept the first pair of peduncles

40. The intervertebral stabilizing device according to claim 39, wherein the first incidence angle ranges between 0 and 20 degrees.

41. The intervertebral stabilizing device according to claim 40, wherein the first incidence angle is equal to 12 degrees.

42. The intervertebral stabilizing device according to claim 35, wherein the second coupling portion has either a saddle- or a ‘U’-shaped configuration, comprising two second branches so as to be fitted on the second spinous bone according to a shape coupling.

43. The intervertebral stabilizing device according to claim 42, wherein to the second coupling portion is associated the second pair of fixing bars so that the stresses transmitted to the second fastening element are distributed both on the second spinous bone and the second pair of peduncles

44. The intervertebral stabilizing device according to claim 42, wherein the second fixing bars are oriented so that, relative to a plane which is perpendicular to a symmetry plane of the second vertebra are angled of a second incidence angle so as to intercept the second pair of peduncles.

45. The intervertebral stabilizing device according to claim 44, wherein the second incidence angle ranges between 0 and 20 degrees.

46. The intervertebral stabilizing device according to claim 45, wherein the second incidence angle is equal to 12 degrees.

47. The intervertebral stabilizing device according to claim 35, wherein at least one of the first and second fastening elements comprises fastening screws adapted to firmly fasten the fastening elements to the peduncles of the vertebrae.

48. The intervertebral stabilizing device according to claim 47, wherein the fastening screws comprise a bush comprising a notch extending along a diameter of the bush and having a thickness which is not lower than the thickness of the fixing bars so as to house a portion of the fixing bars in order to allow the screws to be coupled with the fixing bars.

49. The intervertebral stabilizing device according to claim 35, wherein the anchoring between the spinous bones and the coupling portions is carried out by means of little strings passing around the spinous bones and through suitable coupling holes provided on the coupling portions.

50. The intervertebral stabilizing device according to claim 35, wherein the coupling portions are fastened to the spinous bones by means of dowels at least partially passing through the coupling portions and the spinous bones.

51. The intervertebral stabilizing device according to claim 35, wherein the junction element is a flexible element adapted to allow rotary, translatory and flexural motions among the fastening elements.

52. The intervertebral stabilizing device according to claim 35, wherein the junction element comprises a cylindrical body provided with a plurality of notches being arranged on the circumference of the latter, the notches having a radial depth which is lower than the radius of the cylindrical body so as to provide a helical spring.

53. The intervertebral stabilizing device according to claim 52, wherein the cylindrical body is provided with a single notch being helically arranged so as to provide a single continuous spiral.

54. The intervertebral stabilizing device according to claim 52, wherein the cylindrical body is provided with a central cylindrical hole.

55. The intervertebral stabilizing device according to claim 54, wherein the central cylindrical hole is provided with a diameter equal to about ⅓ of the outer diameter of the same cylindrical body.

56. The intervertebral stabilizing device according to claim 35, wherein the junction element is a joint of the ball type adapted to allow relative rotary motions among the same elements.

57. The intervertebral stabilizing device according to claim 56, wherein the joint of the ball type comprises a spring elastic element therein, so as to dampen the relative motion between the fastening elements.

58. The intervertebral stabilizing device according to claim 35, wherein the connecting bars comprise flexible elements so as to ensure further flexibility to the connecting bars, as well as a better adjustment of the connecting bars to the column length morphology.

59. The intervertebral stabilizing device according to claim 58, wherein the flexible elements comprise a cylindrical body provided with a helical continuous groove and a central hole, being coaxial with the cylindrical body.

60. A medical kit for the intervertebral stabilization comprising
an intervertebral device, and
a template tool for assembling the device the template tool comprising a first and a second measuring elements each adapted to be interfaced with the spinous bones of the two adjacent vertebral bodies each of the measuring elements comprising a saddle portion adapted to be fitted on its respective spinous bone, and a pair of slotted links symmetrically arranged relative to the saddle portion so as to intercept the peduncles of their respective vertebral bodies.

61. The medical kit according to claim 60, wherein each slotted link is provided with a groove adapted to allow either a point of a drift or a marking tool for marking the peduncles of the vertebrae to pass through.

62. The medical kit according to claim 60, wherein the measuring elements can be axially adjusted to each other, along a mutual extension axis Y by means of a screwdriver a connecting portion between the two measuring elements being provided with a graduated scale, in order to provide a direct measuring of the size of the device which is more easily adapted to the column length.

63. The medical kit according to claim 60, comprising a gripper having a pair of grip elements slidingly associated on each other at a first end and each is provided with a pair of tines at a second end being opposite to the first end adapted to grasp the device at the junction element.

64. The medical kit according to claim 60, wherein the gripper is provided with a gauged screw adapted to adjust the axial distance between the two grip elements which can relatively slide on each other.

65. An intervertebral stabilizing method, comprising the steps of:
evaluating the morphology of the column length to be stabilized, by measuring the distance between two spinous bones of two vertebrae adjacent to each other,
marking the zone of the peduncles on which the device requires to be fastened,
fastening the fastening screws on the peduncles so as to orientate the notches of the bushes in a parallel manner to the final arrangement which the connecting bars of the fastening elements will have to take,
grasping and fitting the device between the vertebral bodies, by inserting the bars of the first and second fastening elements into their respective notches of the bushes and by inserting the coupling portions of the device onto the corresponding spinous bones.

66. The intervertebral stabilizing method according to claim 65, comprising the step of final locking of the device bars by inserting and screwing the caps onto the bushes.

67. The intervertebral stabilizing method according to claim 65, wherein the evaluating step of the morphology of the column length to be stabilized, is carried out by fitting a template tool provided with two measuring elements between the spinous bones of the vertebrae and by adjusting the axial position between the two measuring elements.

68. The intervertebral stabilizing method according to claim 67, wherein the marking step of the peduncles is carried out with the aid either of a drift or a marker, by inserting the latter through grooves provided on slots of the template tool.
